(19) United States
(12) Patent Application Publication
(10) Pub. No.: US 2007/0059739 A1
(43) Pub. Date: Mar. 15, 2007

(54) Pure miRNA Sample Preparation Method

(75) Inventor: Kai Qin Lao, Pleasanton, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: APPLERA CORPORATION, Foster City, CA (US)

(21) Appl. No.: 11/458,077

(22) Filed: Jul. 17, 2006

Related U.S. Application Data
(60) Provisional application No. 60/716,633, filed on Sep. 12, 2005.
(60) Provisional application No. 60/780,927, filed on Mar. 8, 2006.

(51) Int. Cl. C12Q 1/68; C07H 21/02; C12P 19/34; C12N 1/08

(52) U.S. Cl. 435/6; 435/91.2; 435/270; 536/23.1

(57) ABSTRACT

The present teachings provide novel methods, compositions, and kits for analyzing mature micro RNAs (miRNAs). By taking advantage of the observation that most mature miRNAs in cells are tightly associated with RISCs, the present teachings provide approaches for studying mature miRNAs without the complications of additional nucleic acids. For example, in some embodiments the present teachings provide a method of purifying mature miRNAs comprising heating a sample to form a lysate, and, degrading the additional nucleic acids. The resulting mixture lacks the additional nucleic acids, and contains mature miRNAs associated with RISCs. Liberating the mature miRNAs from RISCs, for example by a protease, a detergent, and/or heat, can result in a pure collection of mature miRNAs.

### CROSS REFERENCE TO RELATED APPLICATIONS

This application claims a priority benefit under 35 U.S.C. §119(e) from U.S. Patent Application No. 60/716,633 filed Sep. 12, 2005 and U.S. Patent Application No. 60/780,927 filed Mar. 8, 2006, which are incorporated herein by reference.

### FIELD

The present teachings relate to methods, compositions, and kits for purifying mature miRNAs.

### INTRODUCTION

Analysis of expressed nucleic acids can be difficult in small and limited samples. Approaches that multiplex nucleic acid analyses are of growing importance in the biomedical research community. Micro RNAs (miRNAs) are increasingly recognized class of nucleic acids that play important roles in human disease, including cancers (see Lu et al., 2005, Nature 435: 834-838).

The detection of very small nucleic acids (e.g. miRNAs) can be difficult with conventional PCR, due to for example overlapping primers on the short target producing primer dimer artifacts. Recently, techniques that take advantage of the sensitivity, specificity, and dynamic range of quantitative real-time PCR have been developed that allow for the quantitation of such short nucleic acids (see for example U.S. Non-Provisional patent applications Ser. No. 10/881,362 to Brandis et al., Ser. No. 10/944,153 to Lao et al., Ser. No. 10/947,460 to Chen et al., and Ser. No. 11/142,720 to Chen et al.).

Previous work has shown that synthesized siRNA/miRNA is tightly associated with RNA-induced Gene Silencing Complexes (RISCs) in vitro. However, it is unknown if the endogenous miRNAs are also stably bound to RISC complexes in vivo in cells under physiological conditions. Understanding the answer to this question has important implications for understanding the basis for stability and metabolism of miRNAs in living cells, and provides novel avenues for experimental approaches to query these and other issues of miRNA biology.

Based on previous work, it is known that the primary miRNAs (pri-miRNAs) are first cut by Drosha into 70-100 nt miRNA precursors (pre-miRNAs), which are transported from the nucleus into the cytoplasm by exportin5 (Lee et al., Nature, 425, 415-410, and Lund et al., Science, 303, 95-98.). These pre-miRNAs are then processed by Dicer into mature miRNAs, after which they enter into the RISC complex to function (Kim, Nat. Rev. Mol. Cell Bio., 6, 376-385, Zamore et al., Science, 309, 1519-1524, Pham et al., Cell, 117, 83-94, and Pham et al., J. Biol. Chem., 280, 39278-39283). It has been shown that RISCs bind siRNA/miRNA very stably in vitro. In fact, even 2.5M NaCl or 1M Urea cannot dissociate siRNA from RISC complex during purification of the RISC complex from 293T cells (Liu et al., Science, 305, 1437-1441). Martinez et al., also showed that during affinity column purification of RISCs, siRNA bind tightly to them even when treated with 2.5M KCl (Martinez et al., Genes Dev. 18, 975-980). Furthermore, Rivas et al., showed that recombinant human Argonaute 2 and siRNA can form RISCs in vitro (Rivas et al., Nat. Struct. Mol. Bio., 12, 340-349). These in vitro constituted minimal RISCs exhibit the core functions that are attributed to RISCs.

A conservative estimate predicts that there are about 250-300 miRNAs, and their average copy number ranges from between 1000 and 2000, while the highest expression of miRNAs is in the range of 50,000 copies per cell (Lim et al., Science, 299, 1540, Lim et al., Genes Dev. 17, 991-1008, and Chen et al., Nucleic Acids Res., 33, e179). If all the miRNAs are associated with RISC complexes in the cytoplasm, then the number of total RISC complexes in a single cell is likely to be in the order of between 250,000 and 500,000 (Chen et al., Nucleic Acids Res., 33, e179, and Shingara et al., RNA, 11, 1461-1470).

Despite intensive biochemical analysis of miRNA and RISC, it is still not known what proportion of miRNA in vivo are associated with RISCs, and what proportion of miRNAs are ‘free’ from RISCs under physiological conditions in a cell. There are several difficulties that hinder investigations on this question. First, it is difficult to distinguish between pre-miRNAs (double-stranded hairpin of 70-100nt) and mature miRNAs (single stranded of 18-23nt) present in the cell in vivo. Second, biochemical analysis in vitro of small RNAs usually requires loading of radio-labeled double-stranded siRNAs or artificial miRNAs that perfectly complement double-stranded RNA. This procedure adopted in vitro may not correspond to the way in which miRNA are integrated into RISCs from Pre-miRNA that partially complements hairpin RNA. Thirdly, even if the radio-labeled pre-miRNAs used in the biochemical assay faithfully represents the real pre-miRNA in vivo, their concentration used is much higher than the endogenous levels, so that this analysis may not reflect the real situation under physiological conditions. Thus, even after several years of intensive research, it is still unknown if most miRNAs are associated with RISCs, and if so, whether they are tightly associated under physiological conditions in a cell. The answer to this question is critical to understanding, for example, the stability and half-life of miRNAs in vivo. This should also provide important indications concerning miRNA metabolism and disposal in living cells. It could also help to evaluate the effects and duration of siRNA knockdown, an approach that is being widely used for the analysis of gene functions (Dykxhoorn et al., Nat. Rev. Mol. Cell Biol., 4, 457-67).

### SUMMARY

The present teaching provide a method of purifying mature miRNAs in a sample comprising mature miRNAs and additional nucleic acids, said method comprising; lysing the sample; degrading the additional nucleic acids; and, liberating the mature miRNAs. Compositions and kits are also provided. For example, in some embodiments the present teachings provide a composition comprising a collection of RISC-protected mature miRNA, a collection of additional nucleic acids, and at least one experimentally-added active nuclease. In some embodiments, the present teachings provide a composition comprising a collection of RISC-protected mature miRNA and at least one experimentally-added nuclease that is inactivated. Finally, the present teachings provide a kit for purifying miRNAs comprising; at least one nuclease; and, at least one control nucleic acid.

### DRAWINGS

FIG. 1 depicts illustrative data according to some embodiments of the present teachings.

FIG. 2 depicts illustrative data according to some embodiments of the present teachings.

FIG. 3 depicts illustrative data according to some embodiments of the present teachings.

FIG. 4 depicts illustrative data according to some embodiments of the present teachings.

### DESCRIPTION OF SOME EXEMPLARY EMBODIMENTS

It is to be understood that both the foregoing general description and the following detailed description are exemplary and explanatory only and are not intended to limit the scope of the current teachings. In this application, the use of the singular includes the plural unless specifically stated otherwise. Also, the use of “comprise”, “contain”, and “include”, or modifications of those root words, for example but not limited to, “comprises”, “contained”, and “including”, are not intended to be limiting. The term and/or means that the terms before and after can be taken together or separately. For illustration purposes, but not as a limitation, “X and/or Y” can mean “X” or “Y” or “X and Y”.

The section headings used herein are for organizational purposes only and are not to be construed as limiting the described subject matter in any way. All literature and similar materials cited in this application, including, patents, patent applications, articles, books, treatises, and internet web pages are expressly incorporated by reference in their entirety for any purpose. In the event that one or more of the incorporated literature and similar defines or uses a term in such a way that it contradicts that term's definition in this application, this application controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### SOME DEFINITIONS

As used herein, the term “RISC-protected mature miRNAs” refers to a collection of miRNA species that are complexed with RISC molecules, and hence resistant to degradation. These RISC-protected mature miRNAs have already undergone processing, and are not pri-miRNAs or pre-miRNAs. Without being limited to any particular theory, RISC-protected mature miRNAs can be bound directly to RISC, bound to RISC through an intermediary(s), or both.

As used here, the term “liberating the mature miRNA” refers to a process whereby mature miRNAs are released from the RISC complex, thus forming a plurality of free, single-stranded mature miRNAs. The process of liberating can comprise any of a variety of methods known by those of skill in the art to release nucleic acids from proteins. For example, liberating can comprise applying heat, for example 95 C for 5 minutes. In some embodiments, liberating can comprise applying heat, for example 80 C or higher for 5 minutes. Of course, routine experimentation can yield other times and temperatures suitable for heat-based liberating of mature miRNAs. Liberating can comprise treating with a detergent, for example 10% SDS. Liberating can also comprise treating with a chaotropic salt, such as for example guanidinium-based compounds.

As used herein, the term “additional nucleic acids” refers to a collection of nucleic acids that are not mature miRNAs. Included in the term additional nucleic acids are molecules such as pri-miRNAs and pre-miRNAs, as well as other non-coding RNAs, messenger RNAs, transfer RNAs, ribosomal RNAs, and genomic DNA.

As used herein, the term “pure mature miRNAs” refers to a collection of mature miRNAs that are free of additional nucleic acids, are no longer associated with RISC, and are 18-23 nucleotides in length.

As used herein, the term “experimentally-added active nuclease” refers to a nuclease, such as for example an RNAse and/or a DNAse, which is not present endogenously in a sample, but rather is added by an experimentalist. The nuclease is active, in that it can degrade, for example, additional nucleic acids.

As used herein, the term “experimentally-added nuclease that is inactivated” refers to a nuclease, such as for example an RNAse and/or a DNAse, which is not present endogenously in a sample, but rather is added by an experimentalist. The nuclease is originally active, in that it can degrade, for example, additional nucleic acids. The nuclease is later inactivated, for example by treating with heat and/or a protease, thus resulting in an experimentally-added nuclease that is inactivated.

As used herein, the term “heating for a sufficient time and a sufficient temperature to lyse cells and free the additional nucleic acids without liberating mature miRNA” refers to an empirically determined set of time and temperature conditions for a given sample, easily derived by one of skill the art of molecular biology. Such conditions can be measured, by for example, performing a PCR on a target nucleic acid (a messenger RNA) that is desired to be liberated, and ensuring the presence of that target nucleic acid in the lysate, and the absence of that target nucleic in the sample before lysis. Correspondingly, the absence of a free mature miRNA in the lysate, as well as in the unlysed sample, can be determined using an amplification-based assessment. In some embodiments, heating for a sufficient time and a sufficient temperature to lyse cells and free the additional nucleic acids without liberating mature miRNA shall mean that at least 50 percent of the additional nucleic acids are free relative to a non-lysed sample, and that less than 25 percent of mature miRNAs are free relative to a non-lysed sample. In some embodiments, heating for a sufficient time and a sufficient temperature to lyse cells and free the additional nucleic acids without liberating mature miRNA shall mean that at least 75 percent of the additional nucleic acids are free relative to a non-lysed sample, and that less than 10 percent of mature miRNAs are free relative to a non-lysed sample. In some embodiments, heating for a sufficient time and a sufficient temperature to lyse cells and free the additional nucleic acids without liberating mature miRNA shall mean that at least 90 percent of the additional nucleic acids are free relative to a non-lysed sample, and that less than 5 percent of mature miRNAs are free relative to a non-lysed sample. In some embodiments, heating for a sufficient time and a sufficient temperature to lyse cells and free the additional nucleic acids without liberating mature miRNA shall mean that at least 99 percent of the additional nucleic acids are free relative to a non-lysed sample, and that less than 1 percent of mature miRNAs are free relative to a non-lysed sample.

As used herein, the term “detector probe” refers to a molecule used in an amplification reaction, typically for quantitative or real-time PCR analysis, as well as end-point analysis. Such detector probes can be used to monitor the amplification of the target miRNA and/or control nucleic acids such as endogenous control small nucleic acids and/or synthetic internal controls. In some embodiments, detector probes present in an amplification reaction are suitable for monitoring the amount of amplicon(s) produced as a function of time. Such detector probes include, but are not limited to, the 5′-exonuclease assay (TaqMan® probes described herein (see also U.S. Pat. No. 5,538,848) various stem-loop molecular beacons (see e.g., U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons™ (see, e.g., U.S. Pat. Nos. 6,355,421 and 6,593,091), linear PNA beacons (see, e.g., Kubista et al., 2001, SPIE 4264:53-58), non-FRET probes (see, e.g., U.S. Pat. No. 6,150,097), Sunrise®/Amplifluor® probes (U.S. Pat. No. 6,548,250), stem-loop and duplex Scorpion™ probes (Solinas et al., 2001, Nucleic Acids Research 29:E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem Res. Toxicol. 15:118-126; and Yu et al., 2001, J. Am. Chem. Soc 14:11155-11161. Detector probes can also comprise quenchers, including without limitation black hole quenchers (Biosearch), Iowa Black (IDT), QSY quencher (Molecular Probes), and Dabsyl and Dabcel sulfonate/carboxylate Quenchers (Epoch). Detector probes can also comprise two probes, wherein for example a fluor is on one probe, and a quencher is on the other probe, wherein hybridization of the two probes together on a target quenches the signal, or wherein hybridization on the target alters the signal signature via a change in fluorescence. Illustrative detector probes comprising two probes wherein one molecule is an L-DNA and the other molecule is a PNA can be found in U.S. Non-Provisional patent application Ser. No. 11/172,280 to Lao et al., Detector probes can also comprise sulfonate derivatives of fluorescenin dyes with SO3 instead of the carboxylate group, phosphoramidite forms of fluorescein, phosphoramidite forms of CY 5 (commercially available for example from Amersham). In some embodiments, intercalating labels are used such as ethidium bromide, SYBR® Green I (Molecular Probes), and PicoGreen® (Molecular Probes), thereby allowing visualization in real-time, or end point, of an amplification product in the absence of a detector probe. In some embodiments, real-time visualization can comprise both an intercalating detector probe and a sequence-based detector probe can be employed. In some embodiments, the detector probe is at least partially quenched when not hybridized to a complementary sequence in the amplification reaction, and is at least partially unquenched when hybridized to a complementary sequence in the amplification reaction. In some embodiments, probes can further comprise various modifications such as a minor groove binder (see for example U.S. Pat. No. 6,486,308) to further provide desirable thermodynamic characteristics. In some embodiments, detector probes can correspond to identifying portions or identifying portion complements, also referred to as zip-codes. Descriptions of identifying portions can be found in, among other places, U.S. Pat. No. 6,309,829 (referred to as “tag segment” therein); U.S. Pat. No. 6,451,525 (referred to as “tag segment” therein); U.S. Pat. No. 6,309,829 (referred to as “tag segment” therein); U.S. Pat. No. 5,981,176 (referred to as “grid oligonucleotides” therein); U.S. Pat. No. 5,935,793 (referred to as “identifier tags” therein); and PCT Publication No. WO 01/92579 (referred to as “addressable support-specific sequences” therein).

The primers and probes of the present teachings can contain conventional nucleotides, as well as any of a variety of analogs. For example, the term “nucleotide”, as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C₁-C₆ alkyl or C₅-C₁₄ aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT published application nos. WO 98/22489, WO 98/39352;, and WO 99/14226). Exemplary LNA sugar analogs within a polynucleotide include, but are not limited to, the structures: 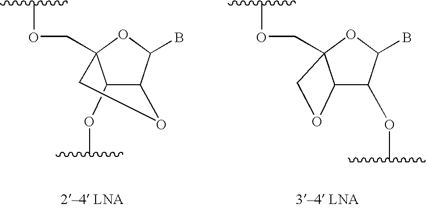
where B is any nucleotide base.

Modifications at the 2′- or 3′-position of ribose include, but are not limited to, hydrogen, hydroxy, methoxy, ethoxy, allyloxy, isopropoxy, butoxy, isobutoxy, methoxyethyl, alkoxy, phenoxy, azido, amino, alkylamino, fluoro, chloro and bromo. Nucleotides include, but are not limited to, the natural D optical isomer, as well as the L optical isomer forms (see, e.g., Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleotide base is purine, e.g. A or G, the ribose sugar is attached to the N⁹-position of the nucleotide base. When the nucleotide base is pyrimidine, e.g. C, T or U, the pentose sugar is attached to the N¹-position of the nucleotide base, except for pseudouridines, in which the pentose sugar is attached to the C5 position of the uracil nucleotide base (see, e.g., Kornberg and Baker, (1992) DNA Replication, 2^{nd} Ed., Freeman, San Francisco, Calif.).

One or more of the pentose carbons of a nucleotide may be substituted with a phosphate ester having the formula: 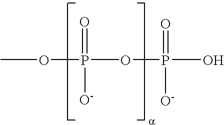
where α is an integer from 0 to 4. In certain embodiments, α is 2 and the phosphate ester is attached to the 3′- or 5′-carbon of the pentose. In certain embodiments, the nucleotides are those in which the nucleotide base is a purine, a 7-deazapurine, a pyrimidine, or an analog thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group may include sulfur substitutions for the various oxygens, e.g. α-thio-nucleotide 5′-triphosphates. For a review of nucleotide chemistry, see: Shabarova, Z. and Bogdanov, A. Advanced Organic Chemistry of Nucleic Acids, VCH, New York, 1994.

Other terms as used herein will harbor meaning based on the context, and can be further understood in light of the understanding of one of skill in the art of molecular biology. Illustrative teachings describing the state of the art can be found, for example, in Sambrook et al., Molecular Cloning, 3rd Edition.

In the present teachings, we employed stem-loop reverse transcription real-time PCR assays (see U.S. patent application Ser. No. 10/947,460 and Chen et al., Nucleic Acids Res. Nov. 27, 2005; 33(20):e179) to detect endogenous miRNAs in a cell. In short, a reverse primer comprising a single-stranded target-specific region complementary to a given miRNA is employed. The reverse primer also contains a double stranded stem, and a single stranded loop. This stem-loop primer is extended in a reverse transcription reaction. Thereafter, a PCR is performed. The reverse primer in the PCR was encoded in the loop of the stem-loop primer, and the forward primer in the PCR comprises a target-specific region, and a non-complementary tail. The accumulation of reaction products in the PCR is measured using a 5′ nuclease detector probe.

These studies verified that this assay is specific to mature miRNAs, and it can easily discriminate between mature miRNA, genomic DNA from which miRNAs originate, primary miRNAs (Pri-miRNAs), and precursor miRNAs (Pre-miRNAs). Furthermore, the assay is very sensitive because it can detect miRNA in pg amounts of total RNA sample. This approach therefore offers a unique opportunity to detect endogenous levels of miRNAs directly and without modifications. As a result of employing this real-time PCR based method to find out what proportion of miRNAs are associated with RISCs in vivo in a cell under physiological conditions, the present teachings provide novel methods, compositions, and kits for studying miRNAs.

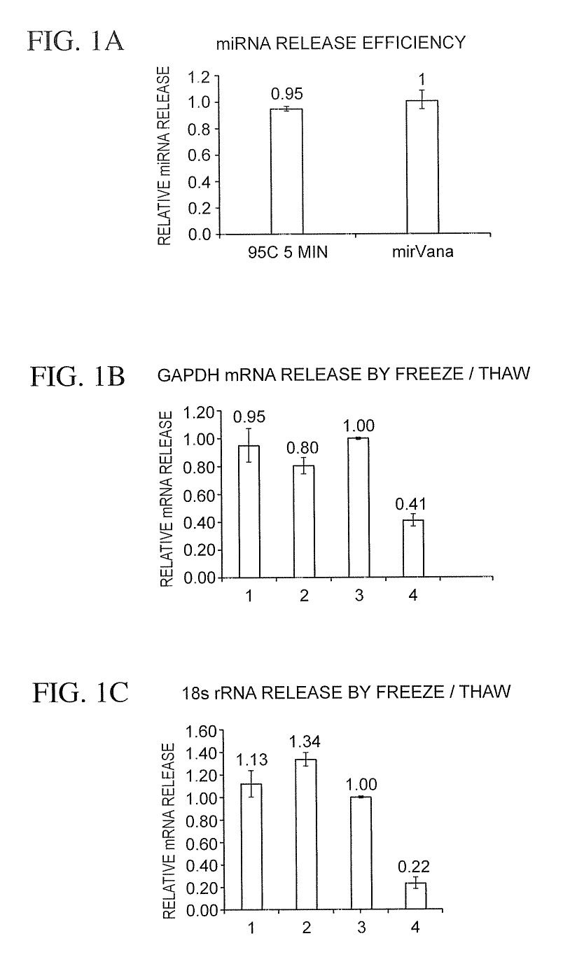

First, we established conditions that release all miRNAs from cells. After a series of tests, we found that treatment of cells at 95 C for 5 minutes can release most if not all miRNAs from ES cells (FIG. 1A). MirVana is a well-known reagent that is highly efficient for the isolation of small RNAs, including miRNAs, from cells or tissues. FIG. 1A shows that treatment at 95 C for 5 minutes and MirVana showed similar efficiency for the release of miRNAs from cells.

Second, we sought to establish a milder condition that can completely release RNA from cells, but without destroying stable protein complexes. From our investigations, we found that subjecting cells to three freeze-thaw cycles lysed ES cells (FIG. 1B), as confirmed by microscopic examination that showed that most cells were lysed after this treatment. We then checked for the release of mRNA of a housekeeping gene, GAPDH that is highly expressed in cells, and found that most of the GAPDH mRNA was released from ES cells after freeze-thawing. Further treatment of the sample at 95 C for 5 minutes did not release any further mRNA. To confirm that this was the case, we also checked for the release of 18s rRNA following this treatment, which confirmed the release of most of the 18s rRNA from ES cells (FIG. 1C). These data show that three freeze-thaw cycles can release most of the free RNAs present in cells. (As shown in FIGS. 1B and 1C, the samples were as follows: (1) three cycles of freeze-thaw followed by incubation at 95 C for 5 minutes; (2) three freeze-thaw cycles; (3) treatment at 95 C for 5 minutes; (4) treatment at 4 C only as a control.).

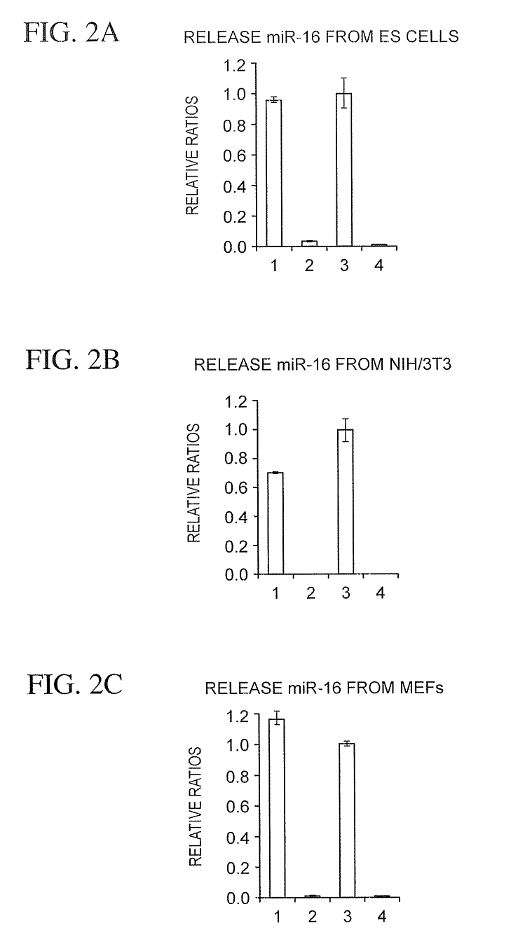

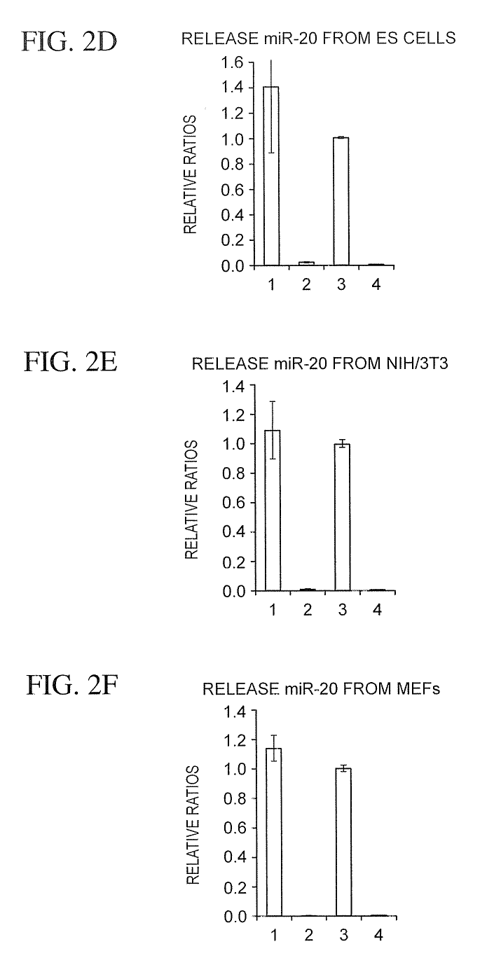

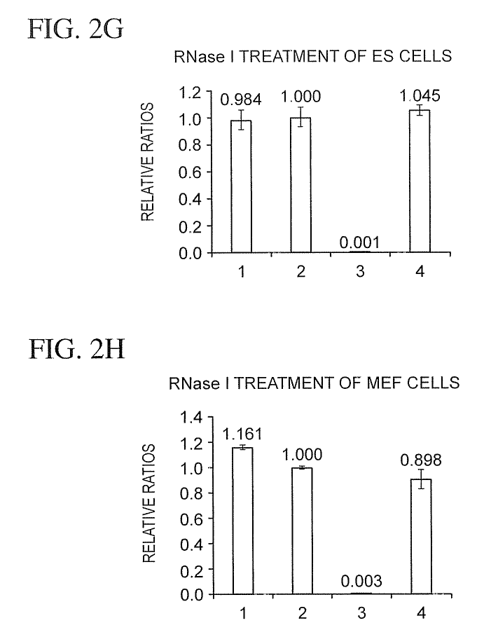

Next, we examined the levels of endogenous miRNA that are associated with RISC complexes in cells by stem-loop RT-PCR. MiR-16 and mir-20 are two highly expressed miRNAs in ES cells. When we compared the levels of miR-16 released following treatment at 95 C for 5 minutes with the levels detected following three freeze-thaw cycles, we found at least 30-fold more miRNA following the first treatment (FIG. 2A). (In FIG. 2A-C, the samples were treated as follows: (1) three freeze-thaw cycles followed by 95 C for minutes; (2) three freeze-thaw cycles only; (3) treatment at 95 C for 5 minutes; (4) treatment at 4 C only as a control.) Thus it seems that the levels of free miR-16 in ES cells are less than 3% of the total miR-16 present in ES cells. In other words, under physiological conditions, most of the miR-16 is stably associated with RISC complexes. We similarly checked the levels of miR-16 in another cell line, NIH/3T3 cells, and in primary mouse embryonic fibroblasts (MEFs). We found that the levels of free miR-16 accounted for at most only 1-2 percent of all miR-16 present in NIH/3T3 cells and MEFs (FIGS. 2B, 2C). To exclude any sequence-specific bias in our analysis, we also examined the levels of miR-20, another highly expressed miRNA, and again found a similar ratio of free versus bound miRNA (FIG. 2D-2F). Therefore, we conclude that under physiological conditions in vivo, most of the miRNAs are tightly associated with RISC complexes, and possibly only 1-3 percent, if any, are free in cells. (In FIGS. 2D-2F, the samples were treated as follows: (1) three freeze-thaw cycles followed by 95 C for minutes; (2) three freeze-thaw cycles only; (3) treatment at 95 C for 5 minutes; (4) treatment at 4 C only as a control.).

To confirm that the miRNAs released by freeze-thaw cycles are indeed free miRNA, we treated the samples with RNase I. RNase I is known to degrade any RNAs into a mixture of mono-, di-, and trinucleotides. First, we confirmed that RNase I treatment at 37 C for 5 minutes can indeed completely degrade free miRNAs. RNase I treatment of miRNA obtained following treatment of cells at 95 C for 5 minutes did indeed degrade all miRNAs to background levels (FIGS. 2G and 2H; compare column 3 and column 4). (In FIG. 2G, the samples were treated as follows: (1) ES cells after three freeze-thaw cycles, were treated with RNase I for 5 minutes, and following exposure at 95 C for 5 minutes to release all RISC-bond miRNAs; (2) three freeze-thaw cycles followed by incubation in the buffer for 5 minutes, and at 95 C for 5 minutes to release all RISC-bond miRNAs (as a control from RNase I treatment); (3) incubation at 95 C for 5 minutes to release miRNAs from RISC complex, followed by RNase I treatment for 5 minutes to release miRNAs from RISC complex, followed by RNase I treatment for 5 minutes, and further incubation of the cell lysate at 95 C for 5 minutes; (4) ES cells were incubated at 95 C for 5 minutes to release miRNAs from RISC complex, followed by treatment with buffer treatment for 5 minutes, and incubation at 95 C for 5 minutes (as control for RNase I treatment). FIG. 2H was treated in an analogous fashion to that depicted in 2G, except the cells were MEFs rather than ES cells.) Second, we found that the samples obtained after three freeze-thaw cycles when treated with RNase I for 5 minutes, did not degrade miRNAs associated with RISC dramatically (FIGS. 2G and 2H; compare column 1 and column 2). Finally, we compared cell lysate containing intact RISCs (following three freeze-thaw cycles) to samples that were treated at 95 C for 5 minutes that destroys the RISC complex. RNase I treatment of these samples showed that less than one percent miRNAs were degraded in samples following the first treatment compared to the latter samples (FIGS. 2G and 2H; compare column 1 and column 3). This demonstrates that nearly all miRNAs are tightly associated with RISC complexes in cells and only a tiny fraction of miRNA are free in intact cells under physiological conditions.

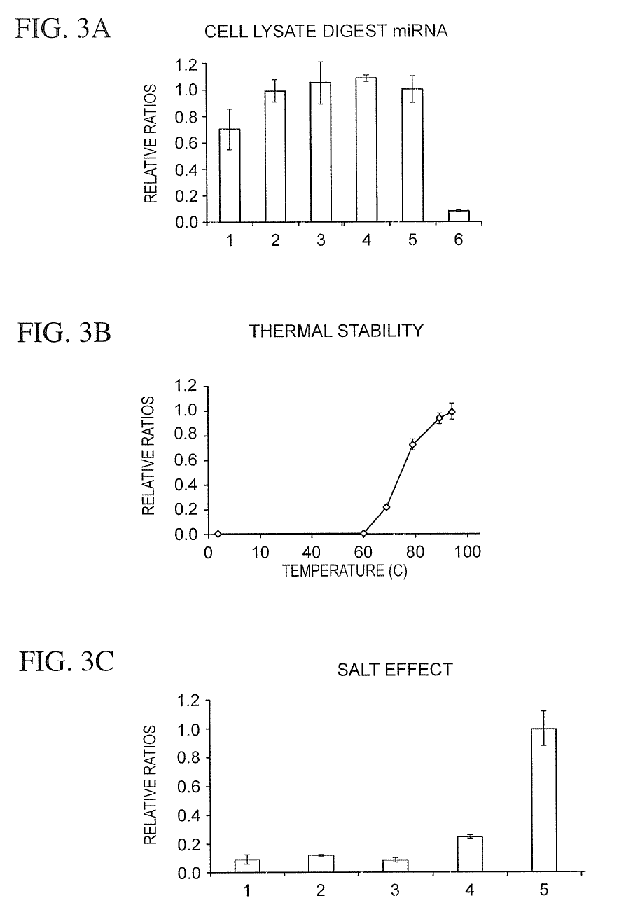

We next considered a possible argument that we may be underestimating free miRNA in cells because any endogenous RNases in cell lysate (but not in living cells) may degrade free miRNAs dramatically. To exclude this possibility, we added purified total RNA (including mature miRNAs) into cell lysate obtained after three freeze-thaw cycles. Our analysis showed that incubating mature miRNA in cell lysate did not cause their degradation to any significant degree (FIG. 3A). This provides evidence that the levels of miRNA in cell lysate are not underestimated in our assay. (In FIG. 3A, the samples were treated as follows: (1) purified total RNA was incubated with the ES cell freeze-thaw cell lysate (6000 cells/ul) for 10 minutes; (2) as above following 1:10 dilution of ES cell lysate; (3) as above following 1:100 dilution of the lysate; (4) as above following 1:1000 dilution of the lysate; (5) purified total RNA as a control; (6) ES cell lysate alone as a control.).

Next, we set out to determine the stability of the miRNA association with the RISC complex. To test this, we treated ES cells at different temperatures and found that miR-16 was stably associated with the RISC complex even after treatment at 60 C for 5 minutes. It was only following treatment of the samples at 80 C for 5 minutes that most of the miR-16s were released from the RISC complexes (FIG. 3B). These observations suggest that most of the endogenous miRNAs are tightly associated with RISC complexes in cells. (in FIG. 3B, the samples were treated at different temperatures for 5 minutes, followed by measurement of miR-16 levels).

It has previously been reported that siRNA can bind stably to RISC complexes even in the presence of 2.5M NaCl, 2.5M KCl, or 1M Urea. We found that the endogenous miR-16 was stably associated with RISC even following treatment with 2.5M NaCl or KCl or ES cell lysates. Treating ES cell lysates obtained following three freeze-thaw cycles with 2.5M NaCl or KCl did not show a dramatic increase in the release of miRNA (FIG. 3C). These data suggest that endogenous miRNAs are tightly bound to RISCs in cells, which resembles the stable association between siRNA/RISC in vitro. (In FIG. 3C, the samples were treated as follows: (1) 2.5M NaCl for 10 minutes; (2) as above but with 2.5M KCl for 10 minutes; (3) control untreated ES cell lysate; (4) 5M NaCl for 10 minutes; (5) treatment of lysate at 95 C for 5 minutes to release all miRNAs.)

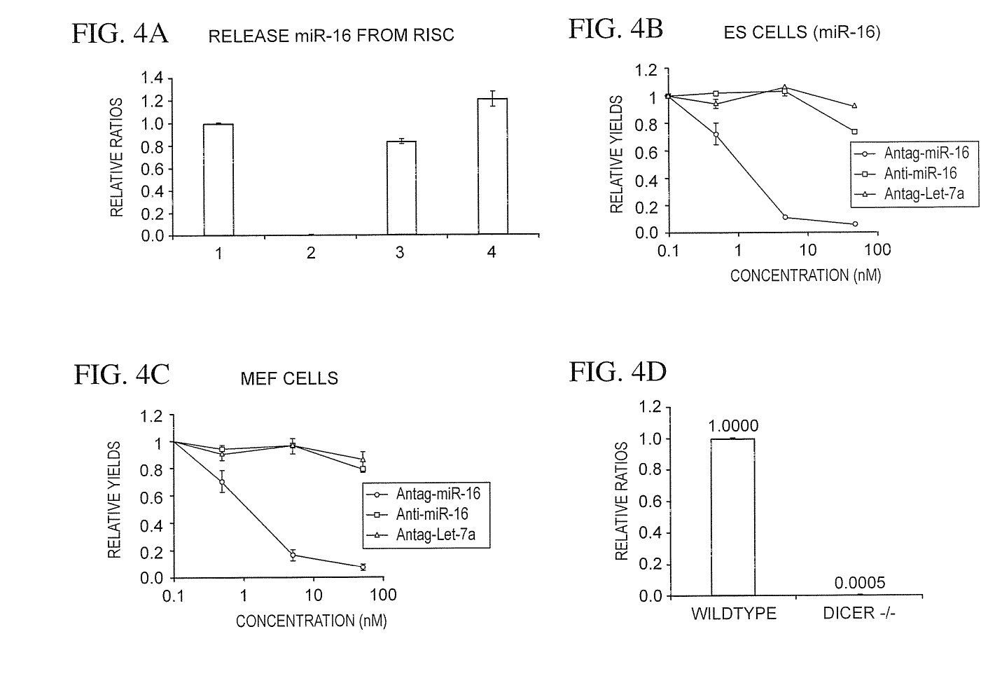

Next, we wanted to assess the dynamics of the association between miRNAs and RISC complexes. To test this, we loaded 0.5uM synthetic Pre-miRNA or mature miRNA into ES cell lysate and incubated the sample for 30 minutes. We then checked the amount of miR-16 associated with RISC in the cell lysate. We found that neither adding Pre-miRNA nor mature miRNA of Let-7a could release miR-16 from RISC complexes (FIG. 4A). (In FIG. 4A, the samples were treated as follows: (1) 95 C for 5 minutes to release miRNAs (positive control); (2) 95 C for 5 minutes followed by incubation with RNase I for 30 minutes to degrade ‘free’ miRNAs, then 95 C for 5 minutes to release RISC-bond miRNAs (as negative control); (3) ES cells subjected to freeze-thaw cycles followed by incubation with 0.5 uM synthesized let-7a for 30 minutes, and then with RNase I for 30 minutes to degrade ‘free’ miRNAs, and finally 95 C for 5 minutes to release RISC-bond miRNAs; (4) ES cell lysate incubated with 0.5 uM synthesized pre-let-7a for 30 minutes, and then with RNase I for 30 minutes to degrade ‘free’ miRNAs, and finally at 95 C for 5 minutes to release RISC-bond miRNAs). Adding 0.5 uM synthetic single-stranded mature miRNAs could not replace endogenous miRNAs in RISC complexes, which indicates that the relative abundance of miRNAs is determined by the amount of initial primary miRNA transcripts. Adding 0.5 uM synthetic hairpin precursor Let-7a (Pre-Let-7a) also could not replace endogenous miRNAs from their RISC complexes. Without intending to be limited by any particular theory, these data suggest the following possibilities. First, the replacement kinetic may be very slow and it may take hours or more to see an effect. Second, precursors may be “transported” into RISC complexes by other protein complexes in living cells. This suggests that the miRNA/RISC association is very stable and free miRNA cannot significantly replace miRNA that is already present in RISC complexes.

Recently, a novel chemically modified oligonucleotide, referred to as ‘antagomirs,’ have been shown to be able to knockdown complementary miRNA by promoting their degradation (Krutzfeldt et al., Nature, 438, 685-689). We proposed that one possibility for this mechanism is that antagomir molecules may bind to target miRNA within RISC and dissociate it from this complex. RNases may subsequently degrade dissociated miRNA/antagomir hybrid (the miRNA strand) released in the cell cytoplasm. To test this possibility, we incubated the ES cell lysate obtained following three freeze-thaw cycles described above, with 0.5 nM-50 nM of antagomirs of miR-16 (antagomir-16) for 30 minutes. The sample was then treated with RNAse I to degrade miR-16 that may have been released from RISC complexes. Finally, RNase I was inactivated and the remaining RISC-bound miRNAs were released by treatment of the ES cell lysate at 95 C for 5 minutes, and their levels were estimated by RT-PCR (FIG. 4B). We found that incubation of the sample with 5 nM of antagomir-16 can degrade 90% of miR-16 in ES cell lysate (the antagomir-16 effect of blocking stem-loop RT-PCR reaction have been corrected by comparing to the same amount of synthetic miR-16 for 0.5-50 nM antagomirs data). As a control for the antagomirs, we used antisense miR-16 RNA molecule, or an unrelated antagomir-let-7a, but neither of them were able to promote the release and subsequent degradation of miR-16 from RISC in the freeze-thaw ES cell lysate. We also checked the antagomir effect in a similar lysate from MEFs, and obtained similar results (FIG. 4C). Without being limited by any particular theory, the data are thus compatible with the notion that antagomirs promote dissociation of specific miRNAs from RISC, and this is followed by their subsequent degradation.

Finally, we verified that the stem-loop RT-PCR is specific to mature miRNA and can discriminate between mature miRNAs from their precursors unequivocally. For this purpose, we measured miR-16 expression in MEFs that lack Dicer. It is known that in such Dicer null cells, mature miRNAs are absent and there is an accumulation of pri-miRNAs and pre-miRNAs (Kanellopoulou et al., Genes Dev., 19, 489-501, and Murchison et al., Proc. Natl. Acad. Sci. USA, 102, 12135-12140). We indeed found that compared to wild-type MEFs, there is virtually no detectable miR-16 (FIG. 4D). These data provide additional evidence that stem-loop RT-PCR does specifically detect mature miRNA, and not the corresponding genomic locus, pre-miRNA, or pre-miRNA.

In general, these data show that in cells under physiological conditions, most miRNAs are tightly associated with RISC complexes and only a small fraction and possibly less than three percent are present outside RISCs. To our knowledge, this is the first time that endogenous miRNAs have been demonstrated to be tightly associated with RISCs in vivo under physiological conditions. These observations may explain why miRNAs are so stable in cells, and it also explains why they exhibit a very long half-life. Our study also shows that antagomirs can effectively and specifically displace miRNA from RISC, which is followed by their degradation.

Experimental Procedures

Reverse Transcription:

1 ul of cell lysate was used as template for a 10 ul reaction. RT reaction is done following manufacture's suggestion by using of ABI high capacity cDNA archive kit (Applied Biosystems, CN: 4322171). The reaction condition is as following: 16 C for 30 min then 42 C for 60 min and finally 85 C for 5 min to inactivate MMLV.

Real-Time PCR Reaction:

0.5 ul of RT product was used for a 10 ul reaction. All reactions were duplicated. TaqMan universal PCR master mix of ABI is used as manufacture suggested. The condition is: 95 C for 10 min followed by 40 cycles of 95 C for 15 sec and 60 C for 1 min. All reactions were run on ABI Prism 7000 Sequence Detection System.

Cell Culture:

ES cells were cultured in Glasgo medium (Plus 15% Fetal Calf Serum (GIBCO) and 1000 U/ml LIF). The cells were kept at undifferentiated state judged from the morphology of colonies and Oct4-GFP expression. NIH/3T3 cells were maintained in 10% FCS DMEM medium. Mouse Embryonic Fibroblasts (MEF) were cultured in DMEM medium (with 10% FCS). They were prepared from E13.5 MF1 mouse embryos.

Cell Lysate:

ES cells were resuspend in PBS at 5000-50,000 cells/ul. Then Freeze/thaw for 3 times on dry ice/Room temperature to lysate ES cells. Under microscope, we confirm that most cells broke following this treatment. All miRNA were released from cells by treating cell suspension at 95 C for 5 min.

Degradation of Free miRNA:

NaCl and Tris-Cl (pH 8.0) were added into cell lysate to final concentration of 100 mM and 10 mM, respectively. Then RNase I was added to 2 U/ul final concentration. They were incubated at 37 C for 30 min to degrade all free miRNAs.

Isolation of Total RNA:

MirVana miRNA isolation kit (Ambion) was used to isolate total RNA (Including miRNAs) according to manufacture's protocol.

Synthesis of Oligos:

All oligos were ordered from Integrated DNA Technologies (IDT, Coralville, Iowa).

Mature Let-7a:

SEQ ID NO:1

5′UGAGGUAGUAGGUUGUAUAGU3′

Pre-Let-7a:

SEQ ID NO:2

5′cₛgₛccaauauuuacgugcuₛgₛcₛuₛa3′

Antisense miR-16 RNA:

SEQ ID NO:3

5′CGCCAAUAUUUACGUGCUGCUA3′

Antagomir-let-7a:

SEQ ID NO:4

5′aₛcₛuauacaaccuacuacₛcₛuₛcₛa3′

The capital letters represent ribonucleotides. The lowercase represents 2′O-methyl ribonucleotides. A subscript ‘s’ represents a phosphorothioate linkage.

Some Applications of the Present Teachings

The finding that most mature miRNAs in cells are bound in the RISC complex provides the basis for the kits, methods, and compositions of the present teachings. For example, there is a desire to profile many mature miRNAs from a single sample, without the complication of contaminating precursor miRNAs. In such a situation, one may employ the following method: treat a sample to lyse the cells; degrade the free RNA by treating with a nuclease such as RNAse I (or heat), wherein mature miRNAs are not degraded due to their association with RISC; denature the RNAse I; provide a primer mix (for example random hexamers) and labeled dNTPs; and, label the miRNAs in a primer extension reaction. The labeled products can then be analyzed, for example on a microarray. In some embodiments, samples with many cells may not need amplification. In smaller samples, the primer extension reaction could be cycled, thus linearly amplifying the miRNAs. Examples of cyling reverse transcription reactions can be found, for example, in U.S. Non-Provisional application to Lao et al., Ser. No. 11/421,460, and U.S. Non-Provisional application Bloch et al., Ser. No. 11/421,319.

Some embodiments the present teachings provide a method of purifying mature miRNAs in a sample comprising mature miRNAs and additional nucleic acids, said method comprising; lysing the sample; degrading the additional nucleic acids; and, liberating the mature miRNAs. In some embodiments, the lysing comprises at least two freeze-thaw cycles. In some embodiments, the lysing comprises heating for a sufficient time and a sufficient temperature to lyse cells and free the additional nucleic acids without liberating mature miRNA. In some embodiments, the time is less than 5 minutes and the temperature is less than 70 C. In some embodiments, the degrading comprises treatment with at least one nuclease. In some embodiments, the at least one nuclease is an RNAse. An RNAse can be helpful, for example, in the degradation of precursor miRNAs not associated with RISC, as well as other RNAs in the cell lysate, such as for example messenger RNAs, transfer RNAs, ribosomal RNAs, and various non-coding RNAs. In some embodiments,the RNAse is RNAse I. In some embodiments, the at least one nuclease is a DNAse. A DNAse can be helpful, for example, in the degradation of genomic DNA present in the cell lysate. In some embodiments, the DNAse is DNAse I. In some embodiments, the at least one nuclease comprises an RNAse and a DNAse. Of course, any of a variety of nuclease are commercially available and can be used in the present teachings, for example as can be purchased from New England Biolabs. Further descriptions of various nuclease, and their use in degrading unwanted nucleic acids, can be found, for example in U.S. patent application Ser. No. 10/982,619, and U.S. Pat. No. 6,797,470.

In some embodiments, the present teachings provide a method of selectively synthesizing complementary nucleic acids to mature miRNAs in a sample, without synthesizing complementary nucleic acids to additional nucleic acids in the sample, said method comprising; lysing the sample to form a collection RISC-protected mature miRNA and a collection of additional nucleic acids; degrading the additional nucleic acids, wherein the RISC-protected mature miRNAs are not degraded; liberating the mature miRNAs of the RISC-protected mature miRNAs to form a collection of pure mature miRNAs; treating the pure mature miRNAs with at least one primer and dNTPs; extending the at least one primer in a primer extension reaction; and, synthesizing complementary nucleic acids to mature miRNAs in the sample without synthesizing complementary nucleic acids to additional nucleic acids in the sample. In some embodiments, at least one of the dNTPs comprises a label. In some embodiments, the primer extension reaction comprises a reverse transcriptase. Various reverse transcriptases are readily available to the molecular biology experimentalist, including for example MMLV and rTth, and various commercially available reverse transcriptases available from New England Biolabs, Applied Biosystems, Ambion, and Stratagene.

In some embodiments, the present teachings provide a method of quantitating a plurality of mature miRNAs from a sample comprising; lysing the sample to form a plurality of RISC-protected mature miRNA and a plurality of additional nucleic acids; degrading the additional nucleic acids, wherein the plurality of RISC-protected mature miRNAs are not degraded; liberating the mature miRNAs from the plurality of RISC-protected mature miRNAs to form a plurality of pure mature miRNAs; treating the plurality of pure mature miRNAs with at least one primer, and dNTPs, wherein at least one of the dNTPs comprises a label; extending the at least one primer in a primer extension reaction; labeling the plurality of pure mature miRNAs from the sample to form a labeled plurality of pure mature miRNAs; hybridizing the labeled plurality of pure mature miRNAs to an array; and, quantitating the plurality of mature miRNA from the sample. In some embodiments, the label comprises a florescent moiety. In some embodiments, the label comprises a digoxygenin. Of course, one of skill in the art can choose from any of a large variety of labels available to the contemporary experimentalist, including florophores, chemiluminescent labels, radioactive moieties, quantum dots, etc. In some embodiments, the array comprises a microarray. In some embodiments, the at least one primer comprises a collection of degenerate hexamers. Reverse-transcription-based labeling of RNA for application to various arrays, and associated analysis methods, are well known in the art, and can be found described, for example, in Nature Genetics, January 1999, Volume 21 No 1s, and, Nature Genetics, December 2002, Volume 32 No 4s. In some embodiments, miRNAs can be detected on LNA arrays, as discussed for example in Castoldi et al., May 2006; 12(5):913-20. Epub Mar. 15, 2006.

In some embodiments, the extension reaction can use unlabeled dNTPs, and the resulting products can then be used as a library to be sequenced, for example using a SAGE (Serial Analysis of Gene Expression) approach on a DNA sequencer, such as a capillary electrophoretic sequencer from Applied Biosystems, or an oil-in-water-emulsion PCR-based sequencing approach as described by Diehl et al., Nat Methods. July 2006; 3(7):551-9. Such a library could also be used to discover novel mature miRNAs by sequencing. Such a library could also be used as a ‘tester’ in a subtractive hybridization-type experiment to discover differentially expressed, and/or novel miRNAs in a tissue of interest.

In some embodiments, the present teachings provide a method of amplifying a plurality of mature miRNAs from a sample comprising; lysing the sample to form a plurality of RISC-protected mature miRNA and a plurality of additional nucleic acids; degrading the additional nucleic acids, wherein RISC-protected mature miRNAs are not degraded; liberating the mature miRNAs of the RISC-protected mature miRNAs to form a plurality of pure mature miRNAs; and, amplifying the pure mature miRNAs. In some embodiments, the amplifying comprises a multiplexed reverse transcription reaction, followed by a PCR. In some embodiments, the PCR is multiplexed. In some embodiments, the reverse transcription and the PCR occur in the same reaction mixture (see for example Yang et al. J Vet Sci. December 2004; 5(4):345-51. In some embodiments, the reverse transcription reaction comprises a stem-loop primer. Examples of stem-loop primers are discussed for example, in U.S. patent application Ser. No. 10/947,460. In some embodiments, the PCR comprises a reverse primer that was encoded by the stem-loop primer, and a forward primer, wherein the forward primer comprises a target-specific portion and a tail portion. Various exemplary encoding schemes depicting the various architectural arrangements of the stem-loop primer, and the reverse primer of the PCR encoded therein, are also found in U.S. patent application Ser. No. 10/947,460.

In some embodiments, the present teachings provide a method of quantitating a plurality of mature miRNAs from a sample comprising; lysing the sample to form a plurality of RISC-protected mature miRNAs and a plurality of additional nucleic acids; degrading the additional nucleic acids, wherein RISC-protected mature miRNAs are not degraded; liberating the mature miRNAs of the RISC-protected mature miRNAs to form a plurality of pure mature miRNAs; and, performing a multiplexed reverse transcription reaction on the plurality of pure mature miRNAs, wherein the multiplexed reverse transcription reaction comprises a plurality of stem-loop primers, to form a plurality of extension products; dividing the plurality of extension products into a plurality of reaction vessels, wherein a PCR can occur in a distinct reaction vessel, wherein a reaction vessel comprises a primer pair, wherein the primer pair comprises a reverse primer that was encoded by a stem-loop primer corresponding to a mature miRNA species, and a forward primer that comprises a target-specific portion for the mature miRNA species, and a tail portion; performing a plurality of PCRs in the plurality of reaction vessels; and, quantitating each mature miRNA species in each PCR by inclusion of a detector probe. In some embodiments, the detector probe is a 5′ nuclease cleavable probe. In some embodiments, the detector probe is Sybr Green. Various exemplary encoding schemes depicting the various architectural arrangements of the stem-loop primer, and the reverse primer of the PCR encoded therein, as well as the extent to which a detector probe can be encoded in the stem-loop primer, are also found in U.S. patent application Ser. No. 10/947,460. Various approaches for performing multiplexed encoding reaction, followed by lower-plex decoding amplification reactions, can be found for example in WO2004/051218 to Andersen and Ruff. In some embodiments, the multiplexed encoding reaction can be a PCR-based pre-amplification reaction, as taught for example in U.S. Pat. No. 6,605,451 to Xtrana, and U.S. Non-Provisional application Ser. No. 11/090,830 to Andersen et al., and U.S. Non-Provisional application Ser. No. 11,090,468 to Lao et al., with a subsequent plurality of lower-plex decoding amplification reactions.

Sample Prep Methods Comprising Solid Supports and Binding Molecules

In some embodiments of the present teachings, a solid support is provided to which binding molecules specific for one or more protein components of miRNP have been attached. The binding molecule-coated solid support can be used to purify and concentrate miRNAs through a process comprising (a) lysing a test sample of cells (for example by freeze-thaw, osmotic shock, exposure to mild detergent, etc), (b) removal of substantially all miRNP from the lysate by contacting the lysate with the binding-molecule-coated support; (c) washing the solid support with buffer to remove substantially all non-miRNP biological material; and (d) removal of substantially pure miRNA from the solid support by washing the support. In some embodiments the washing can comprise a small volume of mild (for example pH 3-5) acid. In some embodiments, the washing can comprise an aqueous solution of PCR-compatible organic cosolvent, detergent, or chaotrope.

The eluted miRNA can be diluted in a downstream reaction, such as a reverse transcription reaction. In some embodiments, the dilution is sufficient to render acid, cosolvent, detergent, and/or chaotrope non-inhibitory. In some embodiments, the miRNA can be recovered from the solid support by heat treatment to denature the miRNP. In some embodiments, the miRNA can be recovered from the solid support by mild protease digestion to fragment the miRNP. When a mild protease is employed, the protease can be inactivated, for example by autolysis, to avoid interference with downstream enzymatic steps.

In some embodiments the binding molecules can be polyclonal antibodies, monoclonal antibodies, single-chain antibodies, aptamers, specific binding polypeptides developed by such methods as phage display and bacterial display, cell-based display methods (as discussed for example in U.S. patent application Ser. No. 10/457,943 to Greenfield), or combinations of these.

In some embodiments, the solid support can be a porous bead, a non-porous bead, a tortuous-path filter membrane, or the inner surface of a small container such as a microtube, microwell, or pipettor tip. In some embodiments, the solid support is a small volume of glass, cellulose, or plastic wool, chemically modified to permit covalent attachment of protein molecules and paced in a small pipettor tip. In some embodiments, the small pipettor tip is a 50 ul tip. In some embodiments, the small pipettor tip is a 200 ul tip. In some embodiments, the small pipettor tip is a 20 ul tip.

It will be appreciated that one of skill in the art, in light of the present teachings, can choose the appropriate binding molecule and solid support from these possibilities, and other readily available options within easy reach of the contemporary molecular biologist.

Some Clinical Research Applications

In some embodiments, the present teachings can be applied in the context of drug-related screening of such antagomirs. For example, model organisms treated with miRNA directed drugs, such as antagomirs, can be further analyzed for the efficiency of knock-down. By recognizing that most miRNAs in cells are complexed with RISC, the present teachings facilitate such approaches by providing a way of delineating between those miRNAs associated with RISC that are destroyed, those miRNAs free in the cells' cytoplasm that survived antagomir treatment, and those miRNAs that remain undestroyed in the RISC complex. Thus, quantifying miRNAs in these different conditions (using for example TaqMan-based PCR as described in U.S. patent application Ser. Nos. 10/947,460 and 11/142,720 to Chen, and U.S. patent application Ser. No. 10/944,153 to Lao) provides tools for the experimentalist interested in the efficiency and mechanism of antagomir action to query these important issues.

For example, in some embodiments the present teachings provide a method of assessing the efficacy of miRNA knock-down with an antagomir comprising; treating a sample with an antagomir for a mature target miRNA; measuring the amount of mature target miRNA that is free; comparing the free mature target miRNAs in the sample with an expectation value; and, assessing the efficacy of miRNA knock-down with the antagomir. In some embodiments, the expectation value can be based on a matched sample not undergoing antagomir treatment. If there is a large difference, the efficacy is high. If there is a small difference, the efficacy is low. In some embodiments, high efficacy is a 50 percent or greater difference, a 60 percent or greater difference, a 70 percent or greater difference, an 80 percent or greater difference, a ninety percent or greater difference, a ninety-five percent or greater difference, or a ninety-nine percent or greater difference. In some embodiments, low efficacy is lower than a 50 percent difference, lower than a 40 percent difference, lower than a 30 percent difference, lower than a 20 percent difference, lower than a 10 percent difference, lower than a 5 percent difference, or lower than a 1 percent difference.

In some embodiments, the present teachings provide a method of assessing the efficacy of miRNA knock-down with an antagomir comprising; treating a sample with an antagomir for a mature target miRNA; measuring the amount of mature target miRNA that results from liberation of mature miRNAs from RISCs; comparing the amount of mature target miRNA that results from liberation of mature miRNAs from RISCs in the sample with an expectation value; and, assessing the efficacy of miRNA knock-down with the antagomir. Of course, consistent with the present teachings, following treatment with the antagomir, the sample can be lysed, additional nucleic acids degraded, and pure mature miRNAs collected by liberation. In some embodiments, the expectation value can be based on the amount of RISC-protected mature miRNAs measured in a matched sample not undergoing antagomir treatment. If there is a large difference, the efficacy is high. If there is a small difference, the efficacy is low. In some embodiments, high efficacy is a 50 percent or greater difference, a 60 percent or greater difference, a 70 percent or greater difference, an 80 percent or greater difference, a ninety percent or greater difference, a ninety-five percent or greater difference, or a ninety-nine percent or greater difference. In some embodiments, low efficacy is lower than a 50 percent difference, lower than a 40 percent difference, lower than a 30 percent difference, lower than a 20 percent difference, lower than a 10 percent difference, lower than a 5 percent difference, or lower than a 1 percent difference.

Some Additional Applications of the Present Teachings

In some embodiments, the miRNAs obtained by the methods of the present teachings can employ recently developed techniques that take advantage of the sensitivity, specificity, and dynamic range of quantitative real-time PCR for the quantitation miRNAs. For example, in some embodiments, a miRNA specific “stem-loop” reverse primer is employed in a primer extension reaction followed by a real-time PCR, wherein the stem-loop primer comprises a self-complementary stem, a loop, and a single-stranded miRNA target specific region, as described for example in U.S. Non-Provisional patent application Ser. No. 10/947,460 to Chen et al., In some embodiments, the miRNAs collected by the present teachings can be further analyzed in highly multiplexed RT-PCR reactions, as taught for example Lao et al., U.S. patent application Ser. No. 11/421,449.

In some embodiments, the miRNAs collected by the present teachings can be further analyzed in RNA-templated OLA-PCR reactions, as taught for example in U.S. Non-Provisional application Ser. No. 10/881,362 to Brandis et al., In some embodiments, the miRNAs collected by the present teachings can be further analyzed in highly multiplexed PCR reactions comprising short linear primers, as taught for example in U.S. Non-Provisional application Ser. No. 10/944,153 to Lao et al., In some embodiments, the present teachings can be used to discover new miRNA biomarkers for various cancers, as well as used for detecting and diagnosing various cancers, as taught for example U.S. Non-Provisional patent application Ser. No. 11/421,456 and Lu et al., 2005, Nature 435: 834-838.

In some embodiments, the miRNA purification methods of the present teachings can be applied to samples comprising degraded nucleic acids, as found for example in paraffin-embedded archived tissues. Additional teachings regarding such tissues, and methods of analyzing nucleic acids therein, can be found in co-filed Lao et al., U.S. Non-Provisional application, claiming priority to U.S. Provisional Application No. 60/699,953.

Certain Exemplary Compositions

In some embodiments, the present teachings provide a composition comprising a collection of RISC-protected mature miRNAs, a collection of additional nucleic acids, and at least one experimentally-added active nuclease. In some embodiments, the RISC-protected mature miRNAs, the additional nucleic acids, and the nuclease result from a lysate. In some embodiments, the lysate results from heating. In some embodiments, the at least one experimentally-added active nuclease is an RNAse.

In some embodiments, the RNAse is RNAse I. In some embodiments, the at least one experimentally-added active nuclease is a DNAse. In some embodiments, the DNAse is DNAse I. In some embodiments, the at least one experimentally-added active nuclease comprises an RNAse and a DNAse.

In some embodiments, the present teachings provide a composition comprising a collection of RISC-protected mature miRNAs and at least one experimentally-added nuclease that is inactivated. In some embodiments, the RISC-protected mature miRNAs, the additional nucleic acids, and the nuclease result from a lysate. In some embodiments, the lysate results from heating. In some embodiments, the at least one experimentally-added nuclease that is inactivated is an RNAse. In some embodiments, the RNAse is RNAse I. In some embodiments, the at least one experimentally-added nuclease that is inactivated is a DNAse. In some embodiments, the DNAse is DNAse I.

In some embodiments, the at least one experimentally-added nuclease that is inactivated comprises an RNAse and a DNAse.

Certain Exemplary Kits

The instant teachings also provide kits designed to expedite performing certain of the disclosed methods. Kits may serve to expedite the performance of certain disclosed methods by assembling two or more components required for carrying out the methods. In certain embodiments, kits contain components in pre-measured unit amounts to minimize the need for measurements by end-users. In some embodiments, kits include instructions for performing one or more of the disclosed methods. Preferably, the kit components are optimized to operate in conjunction with one another.

Thus, in some embodiments, the present teachings provide a kit for purifying miRNAs comprising; at least one nuclease; and, at least one control nucleic acid.

In some embodiments, the at least one nuclease is an RNAse. In some embodiments, at least one RNAse is RNAse A. In some embodiments, the at least one RNAse is RNAse I. In some embodiments, the at least one nuclease is a DNAse. In some embodiments, the at least one DNAse is DNAse I. In some embodiments, the at least one nuclease comprises a first nuclease and a second nuclease, wherein the first nuclease is a DNAse and the second nuclease is an RNAse. In some embodiments, the control nucleic is at least one of snoR-U24, snoR-U66, snoR-U19, snoR-U38b, snoR-U49, snoR-Z30, snoR-HelaU6, snoR-U48, snoR-U44, and snoR-U43. In some embodiments, the kit further comprises a PCR primer pair and a detector probe for the at least one control nucleic acid.

As another example, in some embodiments the present teachings provide a kit for purifying miRNAs comprising a nuclease, and a detergent. In some embodiments, the nuclease is an RNAse. In some embodiments, the RNAse is an RNAse A. In some embodiments, the nuclease is DNAse 1. In some embodiments, the kit comprises a first nuclease and a second nuclease, wherein the first nuclease is a DNAse and the second nuclease is an RNAse. In some embodiments, the kit further comprises guanidinium isocyanate. In some embodiments, the kit comprises control miRNAs from a defined source, for example with defined amounts of defined species of miRNAs. Some examples of control nucleic acids appropriate for use in the context of the present teachings can be found for example in U.S. Non-Provisional patent application Ser. No. 11/421,028, and include snoR-U24, snoR-U66, snoR-U19, snoR-U38b, snoR-U49, snoR-Z30, snoR-HelaU6, snoR-U48, snoR-U44, snoR-U43.

In some embodiments, a kit comprises a solid support with an attached binding molecule, and a stem-loop reverse transcription primer. In some embodiments, the kit further comprises a nuclease, a detergent, a PCR master mix, a miRNA specific forward PCR primer, a reverse PCR primer encoded in the stem-loop reverse primer, a reverse transcriptase, a polymerase, nucleotides, a TaqMan low density array comprising pre-spotted miRNA specific forward PCR primer and reverse PCR primer encoded in the stem-loop RT primer, or combinations thereof.

Although the disclosed teachings have been described with reference to various applications, methods, and kits, it will be appreciated that various changes and modifications may be made without departing from the teachings herein. The foregoing examples are provided to better illustrate the present teachings and are not intended to limit the scope of the teachings herein. Certain aspects of the present teachings may be further understood in light of the following claims.

We claim:

1. A method of purifying mature miRNAs in a sample comprising mature miRNAs and additional nucleic acids, said method comprising;
lysing the sample;
degrading the additional nucleic acids; and,
liberating the mature miRNAs.

2. The method according to claim 1 wherein the lysing comprises at least two freeze-thaw cycles.

3. The method according to claim 1 wherein the lysing comprises heating for a sufficient time and a sufficient temperature to lyse cells and free the additional nucleic acids without liberating mature miRNA.

4. The method according to claim 3 wherein the time is less than 5 minutes and wherein the temperature is less than 70 C.

5. The method according to claim 1 wherein the degrading comprises treating with at least one nuclease.

6. The method according to claim 5 wherein the at least one nuclease is an RNAse.

7. The method according to claim 6 wherein the RNAse is RNAse I.

8. The method according to claim 5 wherein the at least one nuclease is a DNAse.

9. The method according to claim 8 wherein the DNAse is DNAse I.

10. The method according to claim 5 wherein the at least one nuclease comprises an RNAse and a DNAse.

11. A method of selectively synthesizing complementary nucleic acids to mature miRNAs in a sample, without synthesizing complementary nucleic acids to additional nucleic acids in the sample, said method comprising;
lysing the sample to form a collection RISC-protected mature miRNA and a collection of additional nucleic acids;
degrading the additional nucleic acids, wherein the RISC-protected mature miRNAs are not degraded;
liberating the miRNAs of the RISC-protected mature miRNAs to form a collection of pure mature miRNAs;
treating the pure mature miRNAs with at least one primer and dNTPs;
extending the at least one primer in a primer extension reaction; and,
synthesizing complementary nucleic acids to mature miRNAs in the sample without synthesizing complementary nucleic acids to additional nucleic acids in the sample.

12. The method according to claim 11 wherein at least one of the dNTPs comprises a label.

13. The method according to claim 11 wherein the primer extension reaction comprises a reverse transcriptase.

14. The method according to claim 11 wherein the lysing comprises at least two freeze-thaw cycles.

15. The method according to claim 11 wherein the lysing comprises heating for a sufficient time and a sufficient temperature to lyse cells and free the additional nucleic acids without liberating mature miRNA.

16. The method according to claim 15 wherein the time is less than 5 minutes and wherein the temperature is less than 70 C.

17. The method according to claim 11 wherein the degrading comprises treating with at least one nuclease.

18. The method according to claim 17 wherein the at least one nuclease is an RNAse.

19. The method according to claim 18 wherein the RNAse is RNAse I.

20. The method according to claim 17 wherein the at least one nuclease is a DNAse.

21. The method according to claim 20 wherein the DNAse is DNAse I.

22. The method according to claim 17 wherein that at least one nuclease comprises an RNAse and a DNAse.

23. A method of quantitating a plurality of mature miRNAs from a sample comprising;
lysing the sample to form a plurality of RISC-protected mature miRNA and a plurality of additional nucleic acids;
degrading the additional nucleic acids, wherein the plurality of RISC-protected mature miRNAs are not degraded;
removing the miRNAs from the plurality of RISC-protected mature miRNAs to form a plurality of pure mature miRNAs;
liberating the plurality of pure mature miRNAs with at least one primer, and dNTPs, wherein at least one of the dNTPs comprises a label;
extending the at least one primer in a primer extension reaction;
labeling the plurality of pure mature miRNAs from the sample to form a labeled plurality of pure mature miRNAs;
hybridizing the labeled plurality of pure mature miRNAs to an array; and,
quantitating the plurality of mature miRNA from the sample.

24. The method according to claim 23 wherein the label comprises a florescent moiety.

25. The method according to claim 23 wherein the label comprises a digoxygenin.

26. The method according to claim 23 wherein the array comprises a microarray.

27. The method according to claim 23 wherein the at least one primer comprises a collection of degenerate hexamers.

28. The method according to claim 23 wherein the lysing comprises at least two freeze-thaw cycles.

29. The method according to claim 23 wherein the lysing comprises heating for a sufficient time and a sufficient temperature to lyse cells and free the additional nucleic acids without liberating mature miRNA.

30. The method according to claim 29 wherein the time is less than 5 minutes and wherein the temperature is less than 70 C.

31. The method according to claim 23 wherein the degrading comprises treating with at least one nuclease.

32. The method according to claim 31 wherein the at least one nuclease is an RNAse.

33. The method according to claim 32 wherein the RNAse is RNAse I.

34. The method according to claim 31 wherein the at least one nuclease is a DNAse.

35. The method according to claim 34 wherein the DNAse is DNAse I.

36. The method according to claim 31 wherein the at least one nuclease comprises an RNAse and a DNAse.

37. A method of amplifying a plurality of mature miRNAs from a sample comprising;
lysing the sample to form a plurality of RISC-protected mature miRNA and a plurality of additional nucleic acids;
degrading the additional nucleic acids, wherein RISC-protected mature miRNAs are not degraded;
liberating the miRNAs of the RISC-protected mature miRNAs to form a plurality of pure mature miRNAs; and,
amplifying the pure mature miRNAs.

38. The method according to claim 37 wherein the amplifying comprises a multiplexed reverse transcription reaction, followed by a PCR.

39. The method according to claim 38 wherein the PCR is multiplexed.

40. The method according to claim 38 wherein the reverse transcription and the PCR occur in the same reaction mixture.

41. The method according to claim 38 wherein the reverse transcription reaction comprises a stem-loop primer.

42. The method according to claim 41 wherein the PCR comprises a reverse primer that was encoded by the stem-loop primer, and a forward primer, wherein the forward primer comprises a target-specific portion and a tail portion.

43. The method according to claim 37 wherein the lysing comprises at least two freeze-thaw cycles.

44. The method according to claim 37 wherein the lysing comprises heating for a sufficient time and a sufficient temperature to lyse cells and free the additional nucleic acids without liberating mature miRNA.

45. The method according to claim 44 wherein the time is less than 5 minutes and wherein the temperature is less than 70 C.

46. The method according to claim 37 wherein the degrading comprises treating with at least one nuclease.

47. The method according to claim 46 wherein the at least one nuclease is an RNAse.

48. The method according to claim 47 wherein the RNAse is RNAse I.

49. The method according to claim 46 wherein the at least one nuclease is a DNAse.

50. The method according to claim 49 wherein the DNAse is DNAse I.

51. The method according to claim 46 wherein the at least one nuclease comprises an RNAse and a DNAse.

52. A method of quantitating a plurality of mature miRNAs from a sample comprising;
lysing the sample to form a plurality of RISC-protected mature miRNAs and a plurality of additional nucleic acids;
degrading the additional nucleic acids, wherein RISC-protected mature miRNAs are not degraded;
liberating the miRNAs of the RISC protected mature miRNAs to form a plurality of pure mature miRNAs; and,
performing a multiplexed reverse transcription reaction on the plurality of pure mature miRNAs, wherein the multiplexed reverse transcription reaction comprises a plurality of stem-loop primers, to form a plurality of extension products;
dividing the plurality of extension products into a plurality of reaction vessels, wherein a PCR can occur in a distinct reaction vessel, wherein a reaction vessel comprises a primer pair, wherein the primer pair comprises a reverse primer that was encoded by a stem-loop primer corresponding to a mature miRNA species, and a forward primer that comprises a target-specific portion for the mature miRNA species, and a tail portion;
performing a plurality of PCRs in the plurality of reaction vessels; and,
quantitating each mature miRNA species in each PCR by inclusion of a detector probe.

53. The method according to claim 52 wherein the detector probe is a 5′ nuclease cleavable probe.

54. The method according to claim 52 wherein the detector probe is Sybr Green.

55. The method according to claim 52 wherein the lysing comprises at least two freeze-thaw cycles.

56. The method according to claim 52 wherein the lysing comprises heating for a sufficient time and a sufficient temperature to lyse cells and free the additional nucleic acids without liberating mature miRNA.

57. The method according to claim 56 wherein the time is less than 5 minutes and wherein the temperature is less than 70 C.

58. The method according to claim 52 wherein the degrading comprises treating with at least one nuclease.

59. The method according to claim 58 wherein the at least one nuclease is an RNAse.

60. The method according to claim 59 wherein the RNAse is RNAse I.

61. The method according to claim 58 wherein the at least one nuclease is a DNAse.

62. The method according to claim 61 wherein the DNAse is DNAse I.

63. The method according to claim 58 wherein the at least one nuclease comprises an RNAse and a DNAse.

64. A composition comprising a collection of RISC-protected mature miRNAs, a collection of additional nucleic acids, and at least one experimentally-added active nuclease.

65. The composition according to claim 64, wherein the RISC-protected mature miRNA, the additional nucleic acids, and the nuclease result from a lysate.

66. The composition according to claim 65 wherein the lysate results from heating.

67. The composition according to claim 64 wherein the at least one experimentally-added active nuclease is an RNAse.

68. The composition according to claim 67 wherein the RNAse is RNAse I.

69. The composition according to claim 64 wherein the at least one experimentally-added active nuclease is a DNAse.

70. The composition according to claim 69 wherein the DNAse is DNAse I.

71. The composition according to claim 64 wherein the at least one experimentally-added active nuclease comprises an RNAse and a DNAse.

72. A composition comprising a collection of RISC-protected mature miRNAs and at least one experimentally-added nuclease that is inactivated.

73. The composition according to claim 72, wherein the RISC-protected mature miRNA, the additional nucleic acids, and the nuclease result from a lysate.

74. The composition according to claim 73 wherein the lysate results from heating.

75. The composition according to claim 72 wherein the at least one experimentally-added nuclease that is inactivated is an RNAse.

76. The composition according to claim 75 wherein the RNAse is RNAse I.

77. The composition according to claim 72 wherein the at least one experimentally-added nuclease that is inactivated is a DNAse.

78. The composition according to claim 77 wherein the DNAse is DNAse I.

79. The composition according to claim 72 wherein the at least one experimentally-added nuclease that is inactivated comprises an RNAse and a DNAse.

80. A kit for purifying miRNAs comprising;
at least one nuclease; and,
at least one control nucleic acid.

81. The kit according to claim 80 wherein the at least one nuclease is an RNAse.

82. The kit according to claim 81 wherein the at least one RNAse is RNAse A.

83. The kit according to claim 81 wherein the at least one RNAse is RNAse I.

84. The kit according to claim 80 wherein the at least one nuclease is a DNAse.

85. The kit according to claim 84 wherein the at least one DNAse is DNAse I.

86. The kit according to claim 80 wherein the at least one nuclease comprises a first nuclease and a second nuclease, wherein the first nuclease is a DNAse and the second nuclease is an RNAse.

87. The kit according to claim 80 wherein the control nucleic is at least one of snoR-U24, snoR-U66, snoR-U19, snoR-U38b, snoR-U49, snoR-Z30, snoR-HelaU6, snoR-U48, snoR-U44, and snoR-U43.

88. The kit according to claim 80 further comprising a PCR primer pair and a detector probe for the at least one control nucleic acid.

89. A method of assessing the efficacy of miRNA knock-down with an antagomir comprising;
treating a sample with an antagomir for a mature target miRNA;
measuring the amount of mature target miRNA that results from liberation of mature miRNAs from RISCs;
comparing the amount of mature target miRNA that results from liberation of mature miRNAs from RISCs in the sample with an expectation value; and,
assessing the efficacy of miRNA knock-down with the antagomir.

90. The method according to claim 89, wherein following treating with the antagomir, the sample is lysed, additional nucleic acids are degraded, and mature miRNAs are collected by liberating.

91. The method according to claim 90 wherein the expectation value is based on the amount of RISC-protected mature miRNAs measured in a matched sample not undergoing antagomir treatment.
